(19) United States
(12) Patent Application Publication
(10) Pub. No.: US 2024/0216186 A1
(43) Pub. Date: Jul. 4, 2024

(54) A BODY-CONFORMING DISPOSABLE PANT-TYPE ARTICLE

(71) Applicant: Essity Hygiene and Health Aktiebolag, Göteborg (SE)

(72) Inventors: Lucas BÄCK, Göteborg (SE); Fanny STENHOLM, Göteborg (SE); Helena WALLIN, Göteborg (SE)

(73) Assignee: Essity Hygiene and Health Aktiebolag, Göteborg (SE)

(21) Appl. No.: 18/557,328

(22) PCT Filed: Jun. 9, 2021
(86) PCT No.: PCT/EP2021/065376

§ 371 Date: Oct. 26, 2023

(51) Int. Cl. A61F 13/49; A61F 13/511; A61F 13/532

(52) U.S. Cl. CPC A61F 13/49017; A61F 13/51108; A61F 13/5323; A61F 2013/49074

(57) ABSTRACT

Pre-tensioned first and second crotch elastic members are arranged extending in the longitudinal direction along the leg edges in the crotch region, the leg edges in the crotch region extending partly or fully in the corresponding crotch elastic member. The crotch region is generally hourglass-shaped with a narrow intermediate portion. The crotch elastic members have first side edges at least partly coinciding with the corresponding leg edge in the crotch region. A distance as measured in the transverse direction between the first side edge of the absorbent core and the first side edge of the first crotch elastic member and a distance as measured in the transverse direction between the second side edge of the absorbent core and the first side edge of the second crotch elastic member varying with at most 30 millimetres along a full length of the crotch elastic members.

### TECHNICAL FIELD

The present disclosure pertains to a disposable absorbent article having crotch leg elastic arranged adjacent an absorbent core in a crotch region of the disposable absorbent article.

### BACKGROUND

In the art of disposable absorbent articles such as disposable pant-type articles and disposable open-type diapers, there are high demands on fit and comfort of the article while at the same time meeting equally high demands on absorbency and leakage protection.

Disposable absorbent articles of this kind are relatively complex products involving multiple components which are combined to form the absorbent article. Such components include nonwoven components, barrier components, elastic components and usually an absorbent core which may be provided in the form of a separately produced absorbent assembly including a barrier layer and a liquid permeable topsheet which together enclose the absorbent core.

It is common to enhance body conformance and leakage security by arranging pre-tensioned elastic elements along all or selected parts of the waist edge and the leg edges of the absorbent article. The pre-tensioned elastic elements tend to gather the web materials to which they are attached and to form ruffled and wrinkled edge portions along the elastic elements. Such ruffled and wrinkled edge portions are undesirable as they may cause chafing especially in the groin area of a user. In addition, wrinkles which are formed perpendicular to a leg edge may serve as channels through which body fluid may leak out of the article. Furthermore, the ruffled and wrinkled edge portions detract from an underwear-like appearance of the absorbent article and may be visible under ordinary clothes.

In disposable absorbent articles comprising an absorbent core, the absorbent core is generally a relatively less flexible component of the absorbent article and may counteract the contractive forces of elastic elements and materials in the article causing the article to be less body conforming in the area of the absorbent core and in the vicinity of the core.

This may be a particular issue in the crotch portion of an absorbent article, where the amount of absorbent material per unit area in the absorbent core usually is greater than in other parts of the core, causing the core to be more resistant to bending in the crotch region. The stiffer and often thicker part of the core which is placed in the crotch region, may affect the ability of leg elastic in the crotch region to form adequate seals against the body of a wearer of the article. The sealing capacity of leg elastic in the crotch region may be improved by increasing the elastic tension in the leg elastic. However, it is undesirable to use crotch elastic elements with high tension, as they may cause discomfort to the wearer such as chafing and red skin markings.

Accordingly, there exists a need for improved crotch elastification of an absorbent disposable absorbent article which is worn in a panty-like fashion.

### SUMMARY

Disposable pant-type articles having good functionality, fit and comfort, may be achieved at least in part by the features of claim 1. Variations of the disclosure are set out in the dependent claims.

Disclosed herein is a disposable pant-type article having a longitudinal direction and a transverse direction, a longitudinal centre line extending in the longitudinal direction, the article comprising:
a front region having a front waist edge and a first and a second longitudinal front side edge;
a rear region having a rear waist edge and a first ad a second rear side edge;
a crotch region being located between the front region and the rear region;
an absorbent core;
a first leg edge and a second leg edge;
a pre-tensioned first crotch elastic member being arranged extending in the longitudinal direction along the first leg edge in the crotch region and a pre-tensioned second crotch elastic member being arranged extending in the longitudinal direction along the second leg edge in the crotch region, the leg edges in the crotch region extending partly or fully in the corresponding crotch elastic member;
an intermediate portion of the crotch region having a smaller width than a width of the crotch region at the front region and a smaller width than a width of the crotch region at the rear region; wherein
each crotch elastic member has a first side edge and a second side edge extending in the longitudinal direction, the first side edge of each crotch elastic member at least partly coinciding with the corresponding leg edge in the crotch region, and each crotch elastic member has a width in an intermediate portion of the crotch elastic member which is smaller than a width of the crotch elastic member at a front end portion of the crotch elastic member and which is smaller than a width of the crotch elastic member at a rear end portion of the crotch elastic member;
the absorbent core has a first and a second side edge, and a first and a second end edge, the first side edge and the second side edge of the absorbent core extending along the leg edges in the crotch region, the absorbent core having a width in an intermediate portion of the absorbent core which is smaller than a width of a front portion of the absorbent core and smaller than a width of a rear portion of the absorbent core, a distance as measured in the transverse direction between the first side edge of the absorbent core and the first side edge of the first crotch elastic member and a distance as measured in the transverse direction between the second side edge of the absorbent core and the first side edge of the second crotch elastic member varying with at most 30 millimetres along the full length of the crotch elastic members, such as with at most 25 millimetres, with at most 20 millimetres or with at most 15 millimetres along a full length of the first crotch elastic member and the second crotch elastic member.

The disposable absorbent article as disclosed herein may be a pant-type article, the first front side edge being joined to the first rear side edge in a first side seam and the second front side edge being joined to the second rear side edge in a second side seam, the front waist edge and the rear waist edge together defining a waist opening, the first leg edge defining a first leg opening and the second leg edge defining a second leg opening.

Alternatively, the disposable absorbent article as disclosed herein may be an open-type diaper which is fastened in a pant-like configuration about the lower part of a wearer's torso.

In the absorbent articles as disclosed herein, the side edges of the absorbent core and the first side edges of the crotch elastic members are shaped side edges such that the crotch region of the absorbent article has a general hour-glass shape. The shape of the side edges of the absorbent core conforms generally to the shape of the first side edges of the crotch elastic members. The absorbent core is arranged in the absorbent article with the portion of the absorbent core having the smallest width being placed in the portion of the crotch region having the smallest width. As the leg edge contour of the crotch elastic members is adapted to the contour of the side edges of the absorbent core, the width of the elasticated area formed between the side edges of the absorbent core and the first side edges of the crotch elastic members which is elasticated by the crotch elastic members is the same or substantially the same along the full length of the crotch elastic members or at least along 70% of the length of the crotch elastic members.

The disposable absorbent article as disclosed herein may be a panel article wherein:
the front region comprises or consists of a front body panel being elastically stretchable in the transverse direction, the front body panel having a front panel waist edge, a front panel crotch edge opposite the front panel waist edge and a first and a second longitudinal front panel side edge;
the rear region comprises or consists of a rear body panel being elastically stretchable in the transverse direction, the rear body panel having a rear panel waist edge, a rear panel crotch edge opposite the rear panel waist edge and a first and a second rear panel side edge;
the crotch region is located between the front panel and the rear panel, the crotch region being delimited in the longitudinal direction by the front panel crotch edge and the rear panel crotch edge;
the first front panel side edge is joined to the first rear panel side edge in the first side seam and the second front panel side edge is joined to the second rear panel side edge in the second side seam;
a front leg edge section of each of the first and the second leg edge is arranged in the elastically stretchable front body panel, a rear leg edge section of each of the first and the second leg edge is arranged in the elastically stretchable rear body panel, and a crotch leg edge section of each of the first and the second leg edge is arranged in the crotch region from the front panel crotch edge to the rear panel crotch edge, a total length of each leg edge being the combined length of the front leg edge section, the crotch leg edge section and the rear leg edge section;
the crotch leg edge section of the first and second leg edges extends partly or fully in the corresponding crotch elastic member;
the crotch leg edge section is shaped such that the intermediate portion of the crotch region has a smaller width than a width of the crotch region at the front panel crotch edge and a smaller width than a width of the crotch region at the rear panel crotch edge;
the width of the absorbent core in the intermediate portion of the absorbent core is smaller than a width of the absorbent core at the front panel crotch edge and smaller than a width of the absorbent core at the rear panel crotch edge.

An elastically stretchable front body panel and/or the elastically stretchable rear body panel in an absorbent article as disclosed herein may comprise or consist of an elastic laminate, the elastic laminate comprising at least a first layer of fibrous material and an elastic layer.

The elastic layer may comprise or consist of a layer of elastic film or a layer of elastic nonwoven.

During production of the absorbent articles as disclosed herein, the elastic materials, such as elastic elements, films and web materials are attached to non-elastic, non-stretchable materials with the elastic materials in a tensioned, stretched-out state. In the final absorbent articles, the elastic is relaxed, causing the non-stretchable materials to gather.

The non-stretchable materials in a absorbent article as disclosed herein, limit the stretchability of elasticated parts of the absorbent article such as elastic front and rear panels, leg elastic, waist elastic, etc. All distances and lengths as disclosed herein are measured with the absorbent article in a flattened generally two-dimensional state with all elastic in the article stretched out to the full extent permitted by the non-stretchable components in the article and with all non-stretchable components in the article in a non-gathered state.

In a disposable absorbent article as disclosed herein, the front end portions of each of the first and the second crotch elastic members may overlap with and be attached to the front body panel and/or the rear end portion of each of the first and the second crotch elastic members may overlap with and be attached to the rear body panel.

The crotch elastic members may have a maximum width in the transverse direction of the article in the range of from 15 mm to 50 mm, such as from 20 mm to 40 mm or from 25 mm to 35 mm, and a length in the longitudinal direction of the article in the range of from 150 mm to 350 mm, such as from 170 mm to 320 mm or from 200 mm to 310 mm. The dimensions of the crotch elastic members are measured with the absorbent article in a flat-out state with the side seams broken or cut open.

The width of the front end portion of each crotch elastic member may be equal to the width of the rear end portion of the crotch elastic member and a ratio between the width of the front and rear end portions and the smallest width of the intermediate portion may be from 1:0.5 to 1:0.9.

The crotch elastic members may be flat, band-shaped crotch elastic members and may comprise or consist of elastic film or elastic nonwoven material.

Pre-tensioning of the crotch elastic members may suitably be achieved by attaching the crotch elastic members to the absorbent article with the crotch elastic members being in a stretched state. The crotch elastic member may be applied and attached to the absorbent article with the end portions of the crotch elastic members in a non-tensioned condition.

The non-tensioned end portions of the crotch elastic members may each have a length of from 5% to 8% of the total length of the crotch elastic member, including the length of the end portions and the tensioned intermediate portion between the end portions.

The absorbent core may form part of an absorbent assembly, the absorbent assembly comprising the absorbent core, a liquid permeable upper core cover layer and a lower core cover layer, the absorbent core being enclosed between the upper core cover layer and the lower core cover layer. The absorbent assembly may further comprise a liquid permeable topsheet and a liquid barrier layer, the absorbent core being enclosed between the liquid permeable topsheet and the liquid barrier layer with the upper core cover layer facing the liquid permeable topsheet and the lower core cover layer facing the liquid barrier layer.

The first side edges of the crotch elastic members in a disposable absorbent absorbent article as disclosed herein may be aligned with a first and a second side edge of the absorbent assembly and share a contour with the first and second side edge of the absorbent assembly in the crotch region.

Alternatively, the upper core cover layer may be a liquid permeable topsheet and the lower core cover layer may be a liquid barrier layer.

The upper core cover layer and the lower core cover layer may be connected in a first side seal extending along the first side edge of the absorbent core and in a second side seal extending along the second side edge of the absorbent core. The absorbent core may comprise one or more channels extending in the longitudinal direction at a distance from each of the first side seal and the second side seal. Each of the one or more channels may be free or substantially free from absorbent material. A channel seal may be arranged in the one or more channels, the channel seal joining the upper and lower layers of the core cover within the channel. A seal strength of the channel seal may be higher than a seal strength of each of the first and the second side seals of the absorbent core.

The channel seal or channel seals may be a permanent seal or permanent seals prohibiting separation of the upper and lower core cover layers and thereby restricting swelling in the thickness direction of the absorbent core in the vicinity of the channels. A permanent seal, is a seal which does not break under normal use and handling of the absorbent article. A permanent seal resists the forces arising from swelling absorbent material in the absorbent core and remains intact throughout use of the absorbent article.

Channel seals may be breakable seals or permanent seals or may be partly breakable and partly permanent seals. The side seals may be permanent seals or may be breakable seals which break under influence of the forces exerted on the side seals as the absorbent material in the absorbent core absorbs fluid and expands. Side seals being partly breakable and partly permanent seals are also contemplated for the absorbent articles as disclosed herein. Breakable side seals together with permanent channel seals may interact to cause the absorbent material to expand predominantly in the lateral or transverse direction of the absorbent core and the absorbent article upon absorption of body fluids.

The seal strength of a permanent seal may be 2.5 N/25 millimetres or more, such as 3 N/25 millimetres or more and the seal strength of breakable seals may be 2.0 N/25 millimetres or less, such as 1.5 N/25 millimetres or less.

In an absorbent article as disclosed herein, a length of the one or more channels in the longitudinal direction may be less than a length in the longitudinal direction of each of the first crotch elastic member and the second crotch elastic member.

The side seals and the channel seal(s) may be adhesive seals, weld seals, or any combination of adhesive seals and weld seals.

Breakable seals may be seals having very low integrity, such as mechanical seals having been formed e.g., by needling or embossing the upper and lower core layers together.

The first side seal, the second side seal and each channel seal may be adhesive seals. The seal strength of the seals may be controlled by varying the basis weight of the applied adhesive in the seals. A basis weight of adhesive in each channel seal may be higher than a basis weight of adhesive in each of the first side seal and the second side seal. The basis weight of adhesive may also be varied along the seals. In a channel seal, the basis weight of the applied adhesive may be higher in a longitudinally central part of the channel seal than at one or both end parts of the channel seal, allowing the end parts of the channel seal to break upon swelling of the absorbent material while the central part of the channel seal remains intact. The permanently sealed part of the channel seal or channel seals may extend in a portion of the absorbent core which is arranged on a non-stretchable crotch material, such as on a non-stretchable crotch panel or on a non-stretchable crotch part of an outer cover of the absorbent article such as an absorbent pant article or an absorbent open diaper. The breakable end parts of the channel seal or channel seals may extend in portions of the absorbent core which are arranged on elastic front and rear body panels of the absorbent article.

In an absorbent article as disclosed herein, the basis weight of adhesive in a breakable seal may be 4 g/m² or more and the basis weight of adhesive in a breakable seal may be 3 g/m² or less.

The absorbent core in an absorbent absorbent article as disclosed herein may comprise super-absorbent material, such as from 50% by weight to 100% by weight of superabsorbent material.

The one or more channels may be constituted by two channels extending spaced apart and symmetrically arranged on each side of the longitudinal center line, between the longitudinal centre line and a corresponding one of the first side seal and the second side seal.

The length of each of the one or more channels in the longitudinal direction of the article may be in the range of from 50 millimeter to 400 millimeter, such as from 75 millimeter to 350 millimeter, from 100 millimeter to 300 millimeter or from 150 millimeter to 250 millimeter.

The absorbent core may be a two-dimensionally shaped core. The absorbent core may have a shape with at least one of a first and a second end portion of the core being wider than a central portion of the absorbent core, such as an hourglass shape or a T-shape in the plane defined by the longitudinal direction and the transverse direction.

The hourglass shapes as referred to herein include all configurations of the core where the end portions are wider than the central portion.

The crotch elastic members may have a greater longitudinal extension than channels in the absorbent core, both towards the front portion of the absorbent article and towards the rear portion of the absorbent article.

If channels are arranged in an hour-glass shaped absorbent core having a narrowed intermediate portion, the channels may be positioned in the absorbent core with a displacement towards the front portion of the absorbent article such that the forward extension of the channels from the narrow intermediate portion of the absorbent core is greater than the rearward extension of the channels from the narrow intermediate portion of the absorbent core. The channels may be coterminous or approximately coterminous with the narrowed intermediate portion of the absorbent core at the rear end of the channels.

The crotch elastic members may be positioned with a displacement towards the rear portion of the absorbent article in an hourglass shaped absorbent core having a narrowed intermediate portion. Thereby, the forward extension of the crotch elastic members from the narrow intermediate portion of the absorbent core is smaller than the rearward extension of the crotch elastic members from the narrow intermediate portion of the absorbent core.

The extension of the channels and the elastic members is measured forward and rearward from a transverse line extending across the crotch region at the location where the absorbent core has the smallest width. In case the absorbent core has a narrow portion extending in the longitudinal direction and having a constant smallest width of the absorbent core, the extension of the channels and the elastic members is measured forward and rearward from a transverse line extending across the crotch region and dividing the narrow portion into two parts of equal extension in the longitudinal direction.

The absorbent core may comprise two or more portions having different absorption capacity per unit area. The central portion of the absorbent core may have a greater absorption capacity per unit area than an absorption capacity per unit area of the first end portion and a greater absorption capacity per unit area than an absorption capacity per unit area of the second end portion.

The absorbent core may be a three-dimensionally shaped, profiled core with a central part of the absorbent core being arranged between the first end part of the absorbent core and a second end part of the absorbent core, as seen in the longitudinal direction, the central part of the absorbent core having a greater thickness than a thickness of the first end part and a greater thickness than a thickness of the second end part.

The central part of the absorbent core may have a uniform first basis weight, the first end part may have a uniform second basis weight and the second end part may have a uniform third basis weight. The first basis weight may be equal to the second basis weight. The central part of the absorbent core may be delimited from each of the first end part and the second end part by a corresponding first transition zone and a second transition zone.

The first end part may be a front end part and the first transition zone may be a front transition zone having an extension in the longitudinal direction of from 5 millimetres to 80 millimetres, such as from 5 millimetres to 50 millimetres, such as from 10 millimeter to 30 millimeter, and the second end part is a rear end part and the second transition zone is a rear transition zone having an extension in the longitudinal direction of from 5 millimetres to 80 millimetres, such as from 20 millimetres to 80 millimetres, such as from 30 millimetres to 60 millimeter. The basis weight of the absorbent core preferably changes linearly in the transition zones between the end portions and the central portion. The front transition zone may have a smaller extension in the longitudinal direction than the rear transition zone.

A ratio between the basis weight of the central portion of the absorbent core and the basis weight of the first end portion may be in the range of from 4 to 1.5 and a ratio between the basis weight of the central portion of the absorbent core and the basis weight of the second end portion may be in the range of from 4 to 1.5.

The end portions may have equal basis weight. However, end portions having different basis weights are also conceivable for the absorbent articles as disclosed herein. A front end portion may have a greater basis weight than a rear end portion.

The length of the absorbent core in an absorbent article as disclosed herein is determined as the distance between a point on the first end edge of the absorbent core where the first end edge intersects with the longitudinal center line and a point on the second end edge of the absorbent core where the second end edge intersects with the longitudinal center line, the length of the absorbent core being in the range of from 300 millimeter to 700 millimeter, such as from 350 millimeter to 600 millimeter or from 400 millimeter to 500 millimeter.

In an absorbent core having a central portion with greater basis weight, the central portion may have a length in the order of from 30% to 70% of the length of the absorbent core, such as in the order of from 40% to 60% of the length of the absorbent core or from 45% to 55% of the length of the absorbent core. The length of the central portion may be approximately half the length of the absorbent core.

As disclosed herein the crotch region of the disposable absorbent article may comprises a non-stretchable crotch web material, such as a non-stretchable nonwoven web.

A pant-type article as disclosed herein may have a three-part chassis with a crotch panel which is connected to a front panel along a front panel crotch edge and which is connected to a rear panel along a rear panel crotch edge. The front and rear panels may be made from elastic or elasticized web material or an elastic laminate material, and the crotch panel may be a non-stretchable web material or a non-stretchable laminate material.

Alternatively, the pant-type article may have a unitary chassis having a non-stretchable outer or inner cover web extending the full distance between the front panel waist edge and the rear panel waist edge, the cover web constituting a non-stretchable layer of each of the front panel and the rear panel and constituting the crotch web material in the crotch region of the pant-type article.

An elastically stretchable front body panel and an elastically stretchable rear body panel may comprise or consist of a stretchbonded laminated elastic web material.

Suitable stretch-bonded laminates, may comprise nonwoven material layers or webs such as spunbond, air laid, wet laid, carded, electro spun or meltblown nonwovens. The nonwoven material may be bonded by any suitable technique, such as by needling, hydroentangling, ultrasonic welding, or thermobonding.

The fibers of the nonwoven materials used herein may be man-made fibers, natural fibers or mixtures of man-made and natural fibers. Man-made fibers include mono-component, bi-component and multicomponent fibers of polymers such as polyolefins, polyesters, polyacrylates, etc., as well as regenerated fibers such as viscose fibers and modal fibers. Natural fibres are for instance cellulosic fibres such as pulp fibers, cotton fibers, flax, hemp, etc.

The absorbent articles as disclosed herein may have an elastic waist feature arranged along all or part of one or both of the waist edges. An elastic waist feature may be formed by one or more elastic elements. The elastic waist element or elements may be integral with the front portion and the rear portion or may be applied as a separate waistband which is attached along the waist edges.

The nonwoven web-materials used in the absorbent articles as disclosed herein may comprise thermoplastic material. The nonwoven web-materials will typically be incorporated in joins and seams in the absorbent article and it may be desirable that the nonwoven webs be weldable by heat or by ultrasonic welding processes. Examples of suitable polymers for use in the fibrous nonwoven webs as disclosed herein are polyethylene polypropylene and other polyolefin homopolymers and copolymers and polyesters. The weldable nonwoven webs have a high content of thermoplastic component and preferably contain at least 50% thermoplastic fibres and more preferably at least 80% thermoplastic fibres.

A reinforcing leg elastic feature may be arranged along each leg edge in the rear region of the absorbent article. In a panel article as disclosed herein a first reinforcing elastic feature may be arranged on the elastically stretchable rear body panel web along the rear leg edge section of the first leg edge and a second reinforcing elastic feature may be arranged on the elastically stretchable rear body panel web along the rear leg edge section of the second leg edge. The reinforcing elastic features are supplementary elastic elements which are applied in addition to the elastic material in the elastic body panels.

The first reinforcing elastic feature may extend along a first part of the rear leg edge section of the first leg edge in a direction away from a longitudinal centre line of the absorbent article towards the first side seam and may have an end point on the rear leg edge section of the first leg edge, with a second part of the rear leg edge section of the first leg edge being free from the first reinforcing elastic feature.

The second reinforcing elastic feature may extend along a first part of the rear leg edge section of the second leg edge in a direction away from the longitudinal centre line of the absorbent article towards the second side seam and may have an end point on the rear leg edge section of the second leg edge, with a second part of the rear leg edge section of the second leg edge being free from the second reinforcing elastic feature.

### BRIEF DESCRIPTION OF THE DRAWINGS

The absorbent articles and the method for their production as disclosed herein will be further explained hereinafter with reference to the appended drawings wherein:

The absorbent articles and the method for their production as disclosed herein will be further explained hereinafter with reference to the appended drawings wherein:

FIG. 1 shows a planar view of a stretched-out absorbent pant-type article with an absorbent assembly and with open side seams, as seen from an inner surface of the pant-type article;

FIG. 2 shows an enlarged view of the absorbent assembly in the pant-type article in FIG. 1 with an absorbent core enclosed in a core cover;

FIG. 3a shows a cross section through the absorbent assembly in FIG. 2, taken along the line Ill-Ill when the absorbent core is in a dry state;

FIG. 3b shows a cross section through the absorbent assembly in FIG. 2, taken along the line Ill-Ill when the absorbent core is in a wet state;

FIG. 4 shows the pant-type article in FIG. 1 with the side seams closed and in a non-stretched out condition, as it appears before use.

### DETAILED DESCRIPTION

Different aspects of the present disclosure will be described more fully hereinafter with reference to the drawings. The embodiments disclosed herein can be realized in many different forms and should not be construed as being limited to the aspects set forth herein. It is to be understood that although the absorbent article shown in the Figures is a pant-type article, the absorbent article as disclosed herein may be an open-type diaper.

The drawings are schematic and individual components, such as layers of material are not necessarily drawn to scale. The absorbent article which is shown in the figures is a simplified article, and the article may contain further features, such as barrier cuffs, fasteners, disposal arrangements, etc. It is also to be understood that waist elastic and rear leg elastic are optional features and, as such, may be realized in other ways within the scope of the appended claims. The side seams in a pant-type article, such as the article shown in the figures may be reclosable side seams, and the pant-type article may be provided with fastener elements to provide reclosability of the side seams.

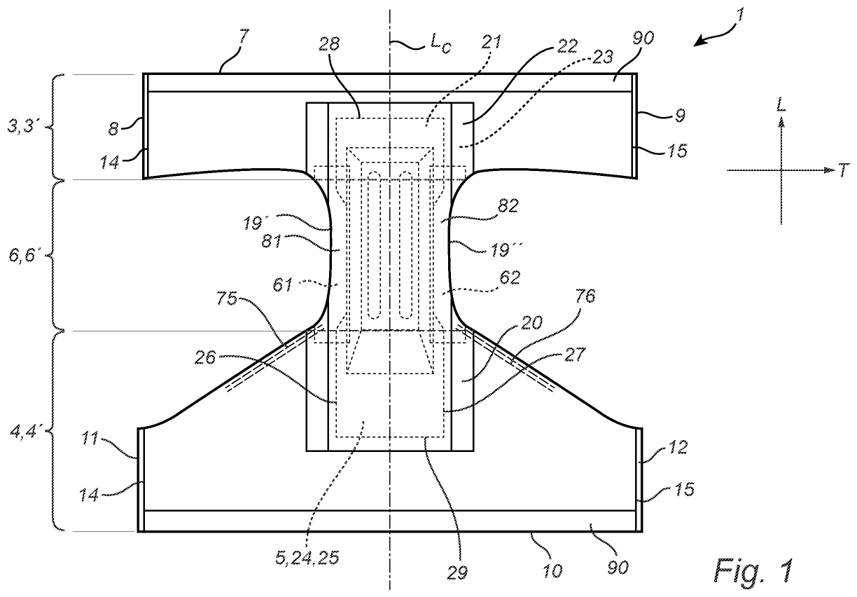

With initial reference to FIG. 1, there is shown an absorbent article 1 in the form of an absorbent incontinence article for adult users. The absorbent article 1 is shown in FIG. 1 in an unfolded and non-gathered condition with all elastic elements in a fully extended state.

The absorbent article has a longitudinal direction L and a transverse direction T, perpendicular to the longitudinal direction with a longitudinal centerline Lc extending in the longitudinal direction L.

The absorbent article 1 is seen from the inner surface which is the surface which will be facing a wearer's body when the article is being worn and which is opposite the outer, garment-facing surface of the absorbent article 1.

The absorbent article 1 comprises a front region 3, a rear region 4 and an absorbent assembly 20 located in a crotch region 6 of the absorbent article 1 and extending in the longitudinal direction L forward in over the front region 3 and rearward in over the rear region 4. The absorbent assembly 20 extends in the longitudinal direction L from the front region 3 through the crotch region 6 and into the rear region 4.

In the absorbent article 1 shown in the figures, the article has a three-part chassis with the front region 3 being formed by an elastic front body panel 3′ the rear region 4 being formed by an elastic rear body panel 4′ and the crotch region 6 being formed by a non-stretchable crotch panel 6′. The crotch panel 6′ is attached to the front body panel 3′ and to the rear body panel 4′. The attachments between the crotch panel and the front and rear panels may be made as overlap seams.

As disclosed herein, the absorbent article 1 may alternatively have any other useful chassis construction, such as a unitary chassis, a two-part chassis or a chassis being constituted by more than three parts. The chassis may include separately attached front and/or rear side flaps.

The absorbent assembly 20 in the absorbent article shown in the figures is a separately produced component which comprises an absorbent core 21. The absorbent core 21 is enclosed between an upper core cover layer 24 and a lower core cover layer 25 of a core cover 5 and which is further enclosed between a liquid permeable topsheet 22 and a liquid barrier layer 23. The absorbent core 21 is arranged between the topsheet 22 and the liquid barrier layer 23 with the upper core cover layer 24 facing the liquid topsheet 22 and the lower core cover layer 25 facing the liquid barrier layer 23.

The front region 3 has a front waist edge 7 extending in the transverse direction T and a pair of front side edges 8,9 extending in the longitudinal direction L. The rear region 4 has a rear waist edge 10 extending in the transverse direction T and a pair of rear side edges 11, 12 extending in the longitudinal direction L.

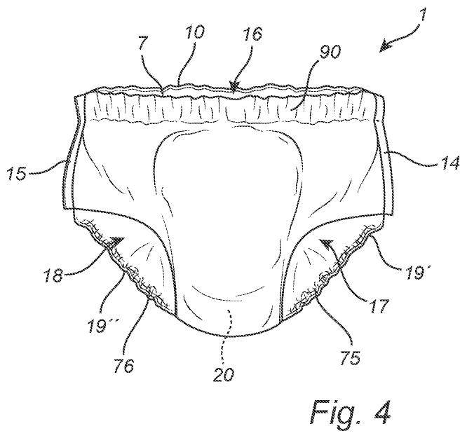

The first front side edge 8 is joined to the first rear side edge 11 in a first side seam 14 and the second front side edge 9 is joined to the second rear side edge 12 in a second side seam 15 to create the pant-type article 1 having a waist opening 16, a first leg opening 17 and a second leg opening 18, as shown in FIG. 4.

The side seams 14, 15 of the absorbent article 1 may be generally band-shaped joins which are formed by ultrasonic welding or thermowelding. To have sufficient strength to withstand the forces to which the absorbent article 1 is exposed during donning of the article and to allow sufficient production tolerances, the side seams commonly have a width in the order of 5 to 10 millimetres. It is also known to make side seams having a width less than 5 mm.

It is generally desired that a soiled absorbent article can be easily removed without having to pull the article down over the legs of a user. Therefore, the side seams are commonly made such that they are breakable by manual force to allow a user or a caregiver to pull apart the side seams before removing a soiled absorbent article.

The waist opening 16 is defined by the front panel waist edge 7 and the rear panel waist edge 10. A first leg edge 19′ defines the first leg opening 17 and a second leg edge 19″ defines the second leg opening 18.

A first pre-tensioned crotch elastic member 61 is arranged extending in the longitudinal direction L along the first leg edge 19′ in the crotch region 6 and a second pretensioned crotch elastic member 62 is arranged extending in the longitudinal direction L along the second leg edge 19″ in the crotch region 6. The crotch elastic members 61, 62 are constituted by band-shaped strips of elastic film or elastic nonwoven which are applied to the pant-type article in a stretched state such that the crotch elastic members 61, 62 are pre-tensioned and form functional leg elastic in the crotch region 6 of the pant-type article 1.

The crotch elastic members 61, 62 have a first side edge 63, 65 and a second side edge 64, 66 extending in the longitudinal direction L. The first side edge 63, 65 of each crotch elastic member 61, 62 at coincides with the corresponding leg edge 19′, 19″ along a major part of the leg edge 19′, 19″ in the crotch region 6.

The leg edges 19′, 19″ are cut into the crotch elastic members 61, 62 in the crotch region 6, whereby the crotch elastic members 61, 62 have a greater width wf, wr in the front and rear end portions 61′, 62′, 61″, 62″ of the crotch elastic members 61, 62 than a smallest width wc of the intermediate portion 61′″, 62′″ of the crotch elastic members.

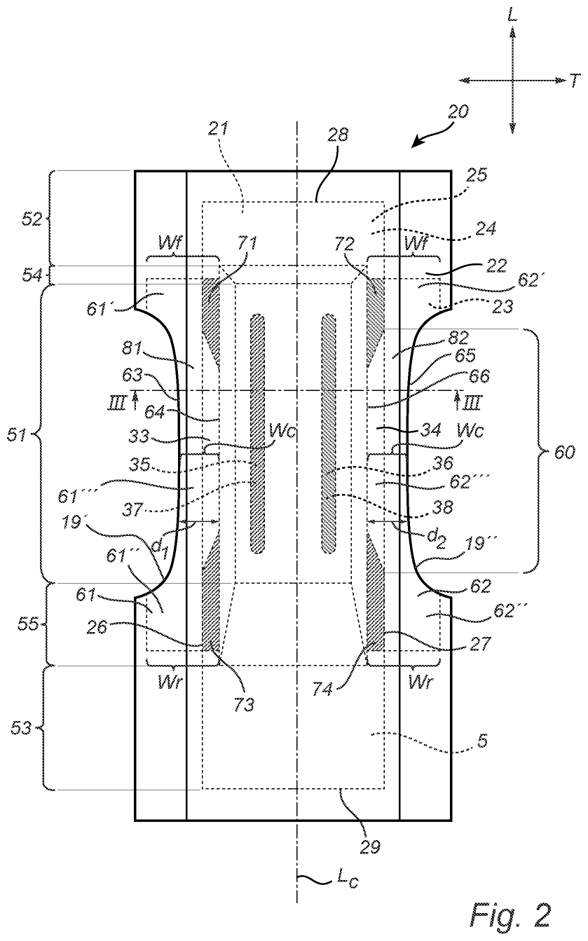

In FIGS. 1 and 2, the leg edges 19′, 19″ are shown with a slightly concave curvature in the crotch region 6, such that also the band-shaped crotch elastic members 61, 62 have a somewhat modified band-shape with a cut-away portion along a part of the leg edges 19′, 19″ in the crotch portion 6.

In the pant-type article 1 which is shown in the Figures, the width wf of the front end portions 61′, 62′ of the crotch elastic members 61, 62 is equal to the width wr of the rear end portion 61″, 62″. The front end portions 61′,62′ and the rear end portions 61″, 62″ have a greater width than the narrowest width of the intermediate portion 61′″, 62′″ of the crotch elastic members 61,62. A suitable ratio between the width of the end portions wf, wr of the crotch elastic members 61,62 and the smallest width wc of the intermediate portion of the crotch elastic members 61,62 may be from 1:0.5 to 1:0.9.

The term “concave edge”, as used herein should be broadly understood as including continuously curved concave edges and edges having an angular shape which changes in one or more distinctly defined steps as well as combinations of a continuous curvature and an angular shape. The concave edge may comprise one or more straight segments and optionally one or more curved segments. A concave crotch leg edge as referred to herein includes V-shaped and U-shaped crotch leg edges as well as any other shape providing the absorbent article with a crotch region which is narrower in a longitudinally intermediate part of the crotch region than at the end parts of the crotch region. A concave edge of a crotch elastic member which has been formed by simultaneous cutting through multiple layers of material in the crotch region, including one or more layers from a peripheral edge portion of the absorbent assembly 20, coincides with a concave edge of the liquid permeable topsheet 22 and with a concave edge of the liquid barrier layer 23 and the absorbent assembly 20.

The width of the front end portions 61′, 62′ of the crotch elastic members 61, 62 is equal to the width of the rear end portions 61″, 62″ of the crotch elastic members 61, 62. A ratio between the width of the end portions 61′, 62′, 61″, 62″ of the crotch elastic members 61, 62 and the smallest width of the intermediate portion 61′″, 62′″ of the crotch elastic members 61, 62 may be from 1:0.5 to 1:0.9.

The end portions 61′, 62′, 61″, 62″ of the crotch elastic members 61, 62 may be non-tensioned parts of the crotch elastic members 61, 61.

With reference to FIG. 2, the absorbent core 21 has an hourglass shape with a first side edge 26 and a second side edge 27, extending along the leg edges 19′, 19″ in the crotch region 6 of the absorbent article 1, a first end edge 28 in the front region 3 of the absorbent article and a second end edge 29 in the rear region 4 of the absorbent article 1.

The first side edge 26 and the second side edge 27 of the absorbent core 21 have a main extension in the longitudinal direction L and the first end edge 28 and the second end edge 29 have a main extension in the transverse direction T.

The absorbent core 21 has a width in a central part 51 of the absorbent core 21 which is smaller than a width of a front end part 52 of the absorbent core and smaller than a width of a rear end part 53 of the absorbent core 21. A distance d1 as measured in the transverse direction T between the first side edge 26 of the absorbent core 21 and the first side edge 63 of the first crotch elastic member 61 and a distance d2 as measured in the transverse direction T between the second side edge 27 of the absorbent core 21 and the first side edge 65 of the second crotch elastic member 62 varies with at most 30 millimetres along the full length of the crotch elastic members 61,62, such as with at most millimetres, with at most 20 millimetres or with at most 15 millimetres.

In the intermediate portions 61′″, 62′″ of the crotch elastic members 61, 62 the shape of the first side edges 63, 65 of the crotch elastic members 61, 62 is defined by the shape of the leg edge 19′, 19″ in the crotch region 6 and the shape of the side edges 26, 27 of the absorbent core generally follows the shape of the leg edges 19′, 19″. In the end portions 61′, 62′, 61″, 62″ of the crotch elastic members 61, 62 where the absorbent core 21 has a greater width, the elasticity in the crotch elastic members 61, 62 is inactivated or at least reduced in front and rear corner portions 71, 72, 73, 74 of the crotch elastic members 61, 62 where the absorbent core 21 overlaps with the crotch elastic members 61, 62.

The absorbent core 21 is arranged in the absorbent article 1 with the portion of the absorbent core 21 having the smallest width being placed in the portion of the crotch region 6 having the smallest width. As the leg edge contour of the crotch elastic members 61, 62 is adapted to the contour of the side edges of the absorbent core 21, the width of an elasticated area between the side edges 26, 27 of the absorbent core and the first side edges 63, 65 of the crotch elastic members 61, 62 which is elasticated by the crotch elastic members is the same or substantially the same along the full length of the crotch elastic members or at least along 70% of the length of the crotch elastic members, if the end portions 61′, 62′, 61″, 62″ of the crotch elastic members 61, 62 are non-tensioned end portions, as disclosed herein. Thus, the absorbent articles as disclosed herein, has a broad, elasticated area 81, 82 with substantially uniform elasticity along the leg edges 19′, 19″ in the crotch region 6. The areas between the leg edges 19′, 19″ and the side edges of the absorbent core 26, 27 which are elasticated by the crotch elastic members 61, 62 are flexible and provides the absorbent article with excellent conformability in the crotch area.

As the contractive forces from the crotch elastic members 61, 62 are distributed over the width of the elasticated area 81, 82, the local pressure exerted against the skin of a wearer is considerably reduced, thereby reducing the risk for chafing or red-marking of the user' skin. Moreover, the wide elasticated area 81, 82 forms a wide gasketing seal against the wearer's skin.

A first channel 35 and a second channel 36 are arranged in the absorbent core 21 and extend in the longitudinal direction Y on either side of the longitudinal centre line Lc and at a distance from each of the first and second side edges 26,27 of the absorbent core 21.

The channels 35,36 are free or substantially free from absorbent material.

The upper core cover layer 24 and the lower core cover layer 25 are connected to each other by a first side seal 33 extending along the first side edge 26 of the absorbent core 21 and by a second side seal 34 extending along the second side edge 27 of the absorbent core 21. In addition, the upper and lower core cover layers 24, 25 are connected by a first channel seal 37 extending in the first channel 35 and by a second channel seal 38 extending in the second channel 36.

As disclosed herein, the seal strength of the first and second channel seals 37,38 is higher than the seal strength of the first and second side seals 33,34 of the absorbent core 21. The seal strength of the first and second channel seals 37,38 may be 2.5 N/25 millimetres or more and the seal strength of the first and the second side seals 33,34 may be 2.0 N/25 millimetres or less.

At least a major part of the channel seals 37,38 are permanent seals which do not break under normal use and handling of the absorbent article. Accordingly, the channel seals 37,38 remain intact or substantially intact even after the absorbent article has absorbed liquid. The side seals 33,34 are shown as breakable seals with low seal strength such that the side seals 33,34 break under influence of the forces exerted of the side seals 33, 34 as the absorbent material in the absorbent core 21 absorbs fluid and expands. The permanent or substantially permanent channel seals 37,38 restrict swelling in the thickness direction of the absorbent article 1, while the breakable side seals 33,34 allow the absorbent material to expand in the lateral or transverse direction. Thus, in the region of the absorbent core 21 where the channels 35,36 are arranged, the combination of permanent or substantially permanent channel seals 37,38 and breakable side seals 33,34 causes the absorbent core 21 to expand predominantly in the transverse direction T upon absorption of body fluid.

It is to be understood that the channel seals can be breakable seals or permanent seals or may be partly breakable and partly permanent seals. The side seals may be breakable side seals or permanent side seals or may be partly breakable and partly permanent seals.

It is also to be understood that the channels are optional features of the absorbent articles as disclosed herein.

As disclosed herein, the side seals 33, 34 and the channel seals 37,38 may be adhesive seals. However, weld seals or combinations of adhesive seals and weld seals are also contemplated for the core cover seals 33,34,37,38.

The upper and lower core cover layers 24, 25 may be formed from a single continuous cover material which is wrapped around the absorbent core 21. In the pant-type absorbent article which is shown in the figures, the upper and lower core cover layers 24,25 are formed from separate webs of material with the absorbent core 21 sandwiched between the core cover layers 24,25.

Depending on the type and size of absorbent article in which the one or more channels as disclosed herein are arranged, the length of the channels may range from 50 millimetres to 500 millimetres.

In a pant-type absorbent article for incontinent adult users, such as the absorbent article 1 shown in the figures, the channels 35,36 are preferably arranged in the crotch region 6 of the article which is the narrow portion of the article which will be placed in the crotch of a user when the article is worn. The narrow crotch region 6 of an absorbent article is the portion of the article which will receive a major part of excreted body fluid, such as urine. The crotch region 6 must therefore have good absorption properties in terms of liquid acquisition, liquid distribution, and absorption capacity. The channels 35,36 in the absorbent core 21 contribute to rapid liquid acquisition and promote fluid distribution by channelling the fluid flow towards the front and rear regions 3, 4 of the absorbent article 1.

In the absorbent article 1 which is shown in the figures, the length of the channels 35,36 substantially corresponds to the length of the crotch region 6 and may typically be in the order of from 150 millimetres to 250 millimetres.

In wearable incontinence articles such as open diapers and pant-type diapers, the channels will typically have a length in the order of from 30% to 50% of the total length of the absorbent core. In smaller absorbent articles such as incontinence shields and sanitary napkins, the channels may extend almost to the ends of the absorbent core, such as up to 80% of the total length of the absorbent core.

In order to enhance the absorbent capacity in the narrow crotch region 6 of the absorbent article 1, the central part 51 of the absorbent core 21 which arranged between a first end part 52 of the absorbent core 21 and a second end part 53 of the absorbent core 21, as seen in the longitudinal direction L, has a greater basis weight than the end parts 52,53. In the absorbent article 1 shown in the figures, the first end part 52 is the front end part 52 of the absorbent core 21 and the second end part 53 is the rear end part 53 of the absorbent core 21.

The central part 51 of the absorbent core 21 has a uniform basis weight and each of the first end part 52 and the second end part 53 of the absorbent core 21 has a uniform basis weight, the central part 51 of the absorbent core 21 being delimited from each of the first end part 52 and the second end part 53 by a corresponding first transition zone 54 and a second transition zone 55. The basis weight of the absorbent core 21 diminishes linearly from the central part 51 of the absorbent core 21 to the end parts 52,53 of the absorbent core 21 within the transition zones 54,55. The rear end part 53 has a greater extension in the longitudinal direction of the absorbent article 1 than the front end part 52. In the absorbent core 21 shown in FIG. 2, the rear transition zone 55 has a greater extension in the longitudinal direction L of the absorbent article 1 than the front transition zone 54.

However, the transition zones may have an equal extension in the longitudinal direction of the absorbent article or the front transition zone may be longer than the rear transition zone. Absorbent cores with a higher basis weight in the central part and lower basis weight in the end parts but without transition zones between the different parts of the cores are also contemplated for the absorbent articles as disclosed herein. Moreover, the central part and the end parts may have non-uniform basis weight.

The ratio between the basis weight of the central part 51 of the absorbent core 21 and the basis weight of the front end part 52 may be in the range of from 4 to 1.5 and the ratio between the basis weight of the central part 51 of the absorbent core 21 and the basis weight of the second end part 53 may be in the range of from 4 to 1.5. The end parts 52, 53 may have the same basis weight or may have different basis weights.

The absorbent core 21 which is shown in the figures is a three-dimensionally shaped core having different thickness in the different parts of the absorbent core. However, it is to be understood that a three-dimensional shape of the absorbent core is optional to the absorbent articles as disclosed herein and that the absorbent core may be planar or substantially planar. In a planar or substantially planar absorbent core, the absorbent capacity in the absorbent core may be different in different parts of the core only as a result of a different absorption capacity per unit area. A difference in absorption capacity per unit area may be achieved e.g., by applying different amounts of superabsorbent material in the different parts of the core and/or by varying the amount of fibres and degree of compression in a fibrous matrix in the absorbent core.

The absorbent core 21 as disclosed herein may comprise any material suitable for absorbing discharged bodily wastes, such as cellulosic fluff pulp, tissue layers, highly absorbent polymers (super absorbents), absorbent foam materials including hydrogel-foam material, absorbent nonwoven materials, or the like. The absorbent core can comprise non-absorbent components such as stiffening elements, shaping elements, binders, etc. Various types of liquid-receiving and liquid distribution elements can also be included in the core.

The absorbent core 21 preferably comprises superabsorbent material in an amount of from 50% by weight to 100% by weight of superabsorbent material, such as from 60% by weight to 100% by weight of superabsorbent material, at least in the central part 51 of the absorbent core 21. As set out herein, the superabsorbent content may be lower in the front end part 52 and/or in the rear end part 53 of the absorbent core than in the central part 51 of the absorbent core 21 or may be the same in all parts of the absorbent core 21.

The superabsorbent material is a polymeric material and may be in the form of particles, granules fibres, flakes, etc.

As disclosed herein, the absorbent core 21 may comprise a mixture of absorbent cellulose fibres, such as cellulose pulp fibres, and superabsorbent material.

A high amount of superabsorbent material in the absorbent core, makes it possible to produce absorbent articles which are thin and discrete while offering high absorbent capacity and an ability to chemically bind and immobilize absorbed fluid which results in an absorbent article having high leakage security and a dry inner surface even after having absorbed a large amount of fluid.

The superabsorbent material absorbs liquid upon swelling and formation of an aqueous gel. In order to fully utilize the absorbent capacity of the superabsorbent material, the absorbent article must provide sufficient expansion room for the superabsorbent material.

As the absorbent core is enclosed in the core cover 5, the swelling superabsorbent material will exert pressure on the core cover. In the central part 51 of the absorbent core 21 where the channels 35,36 with the permanent channel seals 37,38 are arranged, the swelling room is restricted in the area between the channels 35,36 causing this area to grow somewhat thicker and stiffer until the core cover prohibits further swelling.

The absorbent core 21 has an hourglass shape with the distance between the side edges 26,27 of the absorbent core 21 being greater at the end edges 28, 29 of the absorbent core 21 than in an intermediate region 60 of the absorbent core 21 where the absorbent core has a smaller width than at the end edges 28, 29.

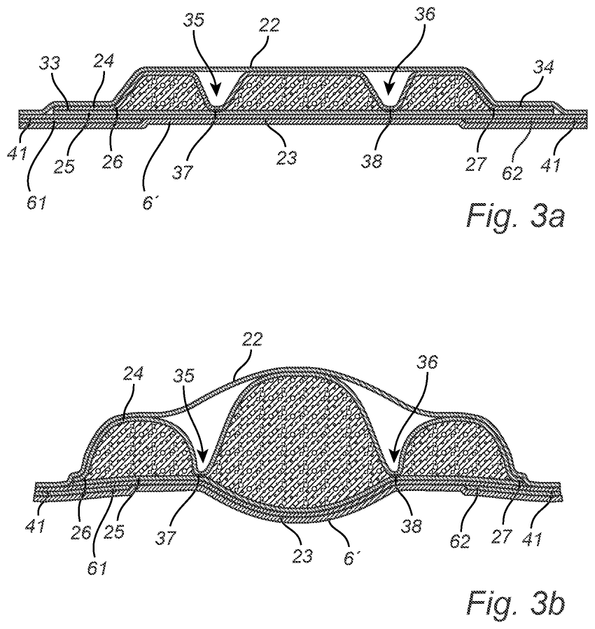

FIGS. 3a and 3b show a cross section taken along the line III-III through the absorbent core 21 in FIG. 2. FIG. 3a shows the absorbent core 21 in a dry state, before the absorbent core 21 has absorbed any liquid and FIG. 3b shows the absorbent core 21 in a wet state, after absorption and swelling of the absorbent core 21.

The absorbent core 21 is shown enclosed between the upper core cover layer 24 and the lower core cover layer 25 and between the liquid permeable topsheet 22 and the liquid barrier layer 23. The absorbent core 21 is arranged between the topsheet 22 and the liquid barrier layer 23 with the upper core cover layer 24 facing the liquid topsheet 22 and the lower core cover layer 25 facing the liquid barrier layer 23.

The topsheet 22 and the liquid barrier layer 23 extend beyond the periphery of the absorbent core 21 and are joined to each other in an edge seal 41 extending along the periphery of the absorbent core outward of the side edges 26, 27 and the end edges 28, 29 of the absorbent core 21. The edge seal 41 between the topsheet 22 and the liquid barrier layer 23 is preferably a permanent seal.

As the absorbent core 21 absorbs body fluid, the absorbent material in the absorbent core 21 swells causing the portion of the absorbent core 21 which is located between the channels 35,36, and which is confined between the permanent channel seals 37,38, to assume a sausage-like shape with the absorbent material completely filling the available space between the channels 35, 36 and causing the topsheet 22 and the upper core cover layer 24 to bulge upwards and the liquid barrier layer 23 and the lower core cover layer 25 to bulge downwards, as shown in FIG. 3b. The absorbent material which is arranged in the portions of the absorbent core 21 which are located between the channels 35,36 with the permanent channel seals 37,38 exerts pressure on the breakable side seals 33,34 causing the side seals 33,34 to rupture and to provide lateral expansion room for the absorbent material. In this way, the absorbent core may absorb liquid and swell while the side edges 26, 27 of the absorbent core 21 remain soft and comfortable. As can be deduced from FIGS. 3a and 3b, the lateral swelling of the absorbent core 21, causes the side edges 26, 27 of the absorbent core to move outward in the transverse direction T, whereby the hourglass shape of the absorbent core becomes gradually obliterated.

The channel seals 37, 38 may be breakable seals at the ends of the channels 35, 36, where the absorbent core 21 has a somewhat greater width than in an intermediate part of the channels 35, 36 where it is desirable to control the direction of swelling such that the portions of the absorbent core 21 which are located along the side edges 26, 27 expand predominantly in the transverse direction of the absorbent core 21, as shown in FIG. 3b.

The length of the one or more channels 35,36 in the longitudinal direction L is shown in the figures to be less than the length in the longitudinal direction L of the crotch elastic members 61.62. The crotch elastic members 61, 62 are shown to be longer than the channels 35, 36, both towards the front portion 3 of the absorbent article 1 and towards the rear portion 4 of the absorbent article 1.

The channels 35, 36 are shown to be positioned in the absorbent core 21 with a displacement towards the front portion 3 of the absorbent article 1. Thereby, the forward extension of the channels 35, 36 from the narrow intermediate portion of the absorbent core 21 is greater than the rearward extension of the channels 35, 36 from the narrow intermediate portion of the absorbent core 21. In FIG. 1, the channels 35, 36 are shown to be approximately coterminous with the narrow intermediate portion of the absorbent core 21 at the rear end of the channels 35, 36.

The crotch elastic members 61, 62 are shown to be positioned in the absorbent core 21 with a displacement towards the rear portion 4 of the absorbent article 1. Thereby, the forward extension of the crotch elastic members 61, 62 from the narrow intermediate portion of the absorbent core 21 is smaller than the rearward extension of the crotch elastic members 61, 62 from the narrow intermediate portion of the absorbent core 21. The extension of the channels 35, 36 and the elastic members 61, 62 is measured forward and rearward from a transverse line extending across the crotch region 6 at the location where the absorbent core 21 has the smallest width. In case the absorbent core 21 has a narrow portion extending in the longitudinal direction L and having a constant smallest width of the absorbent core 21 as is the case in the absorbent article 1 shown in the figures, the extension of the channels 35, 36 and the elastic members 61, 62 is measured forward and rearward from a transverse line extending across the crotch region 6 and dividing the narrow portion into two parts of equal extension in the longitudinal direction L.

The liquid permeable topsheet 22 may comprise or consist of a nonwoven material. Other suitable topsheet materials include tow fibres, porous foams, apertured plastic films and laminates and combinations of such materials. The materials which are best suited as topsheet materials are soft and non-irritating to the skin, are readily penetrated by body fluids, and display low rewet.

The liquid barrier layer 23 may consist of a thin plastic film, e. g. a polyethylene or polypropylene film, a nonwoven material coated with a liquid impervious material, a hydrophobic nonwoven material which resists liquid penetration or laminates of plastic film and nonwoven. The liquid barrier layer material may be breathable to allow vapour to escape from the absorbent body, while still preventing liquids from passing through the liquid barrier layer material.

The topsheet and liquid barrier layer may be connected to each other for example by adhesive bonding, gluing or welding by heat or ultrasonically. The topsheet and/or the liquid barrier layer may further be attached to the core cover by any method known in the art, such as adhesive, heat-bonding, welding, etc.

The pant-type article 1 as disclosed herein may have an elastic waist feature 90 arranged along the waist edges 7, 10. An elastic waist feature 90 may be formed by one or more elastic elements extending parallel with the front waist edge 7 and the rear waist edge 10.

The elastic waist element or elements may be incorporated in the front region 3 and the rear region 4 or may be applied as a separate waistband which is attached to the front waist edge 7 and the rear waist edge 10. The pant-type article 1 which is shown in the Figures has an elastic waist feature 90 which extends around the full circumference of the waist opening 16, as shown in FIG. 4. However, an elastic waist feature may be arranged only along a part of the waist opening, such as only along the rear waist edge, only along the front waist edge or along a part of one or both the front and the rear waist edge which part has a length which is less than the full length of the corresponding waist edge.

A reinforcing leg elastic feature 75, 76 is arranged along each leg edge 19′, 19″ in the rear region 4 of the absorbent article 1. In a panel article as disclosed herein a first reinforcing elastic feature 75 may be arranged on the elastically stretchable rear body panel web along the rear leg edge section of the first leg edge and a second reinforcing elastic feature 76 may be arranged on the elastically stretchable rear body panel web along the rear leg edge section of the second leg edge. The reinforcing elastic features are supplementary elastic elements which are applied in addition to the elastic material in the elastic body panels. The reinforcing elastic features are optional to the absorbent articles as disclosed herein. The reinforcing elastic features 75, 76 which are shown in FIG. 1 are applied only along a part of the rear portion of the leg edges and do not extend the full distance to the side seams 14, 15. However, reinforcing leg elastic features may be applied all the way to the side seams. It is also to be understood that reinforcing leg elastic features may be arranged along the front part of the leg edges.

The elastic material in elastic elements arranged along the leg openings 17,18 and the waist opening 16 as disclosed herein may be any suitable elastic material such as natural or synthetic rubber, thermoplastic elastomers, such as thermoplastic polyurethane or styrene block co-polymers or elastane, also referred as to spandex (polyurethane-polyurea copolymer). The elastic elements may be of the elastane type that is available under the trade name “LYCRA”, but any suitable elastic thread may be used.

The provision of an absorbent core 21 is not limited to absorbent articles having the absorbent core applied to the article as a component of a pre-fabricated absorbent assembly which already comprises a topsheet and a liquid barrier layer.

The disposable absorbent articles as disclosed herein comprise a front region 3, a rear region 4 and a crotch region 6 being located between the front region 3 and the rear region 4 and an absorbent core 21 being arranged in the crotch region 6 and extending in the longitudinal direction into the front and rear regions 3,4 and have a waist edge and a first and a second leg edge 19′, 19″ with pre-tensioned crotch elastic members 61, 62 being arranged extending along the leg edges 19′, 19″ in the crotch region 6, the leg edges 19′, 19″ in the crotch region 6 extending partly or fully in the corresponding crotch elastic member 61, 62. The crotch region 6 is generally hourglass-shaped with a narrow intermediate portion and the crotch elastic members 61, 62 have first side edges 63, 65 at least partly coinciding with the corresponding leg edge 19′, 19″ in the crotch region 6. The absorbent core 21 has first and second side edges 26,27 extending along the leg edges 19′, 19″ in the crotch region 6, the absorbent core 21 is generally hourglass-shaped with a narrow intermediate portion. The distance d1 as measured in the transverse direction T between the first side edge 26 of the absorbent core 21 and the first side edge 63 of the first crotch elastic member 61 and the distance d2 as measured in the transverse direction T between the second side edge 27 of the absorbent core 21 and the first side edge 65 of the second crotch elastic member vary with at most 30 millimetres along the full length of the crotch elastic members 61,62.

1. A disposable absorbent article, the article having a longitudinal direction and a transverse direction, a longitudinal centre line extending in the longitudinal direction, the article comprising:
a front region having a front waist edge and a first and a second longitudinal front side edge;
a rear region having a rear waist edge and a first and a second rear side edge;
a crotch region being located between the front region and the rear region;
an absorbent core;
a first leg edge and a second leg edge;
a pre-tensioned first crotch elastic member being arranged extending in the longitudinal direction along the first leg edge in the crotch region and a pre-tensioned second crotch elastic member being arranged extending in the longitudinal direction along the second leg edge in the crotch region; wherein
the leg edges in the crotch region extend partly or fully in the corresponding crotch elastic member;
an intermediate portion of the crotch region has a smaller width than a width of the crotch region at the front region and a having smaller width than a width of the crotch region at the rear region; wherein
each crotch elastic member has a first side edge and a second side edge extending in the longitudinal direction, the first side edge of each crotch elastic member at least partly coinciding with the corresponding leg edge in the crotch region, and each crotch elastic member has a width in an intermediate portion of the crotch elastic member which is smaller than a width of the crotch elastic member at a front end portion of the crotch elastic member and which is smaller than a width, of the crotch elastic member at a rear end portion of the crotch elastic member;
the absorbent core has a first and a second side edge, and a first and a second end edge, the first side edge and the second side edge of the absorbent core extending along the leg edges in the crotch region the absorbent core having a width in an intermediate portion of the absorbent core which is smaller than a width of a front portion of the absorbent core and smaller than a width of a rear portion of the absorbent core a distance as measured in the transverse direction between the first side edge of the absorbent core and the first side edge of the first crotch elastic member and a distance as measured in the transverse direction between the second side edge of the absorbent core and the first side edge of the second crotch elastic member varying at most 30 millimetres along a full length of the first crotch elastic member and the second crotch elastic member.

2. A disposable absorbent article according to claim 1, wherein the disposable article is a pant-type article, the first front side edge being joined to the first rear side edge in a first side seam and the second front side edge being joined to the second rear side edge in a second side seam, the front waist edge and the rear waist edge together defining a waist opening, the first leg edge defining a first leg opening and the second leg edge defining a second leg opening.

3. A disposable absorbent article according to claim 2, wherein:
the front region comprises or consists of a front body panel being elastically stretchable in the transverse direction, the front body panel having a front panel waist edge, a front panel crotch edge opposite the front panel waist edge and a first and a second longitudinal front panel side edge;
the rear region comprises or consists of a rear body panel being elastically stretchable in the transverse direction, the rear body panel having a rear panel waist edge, a rear panel crotch edge opposite the rear panel waist edge and a first and a second rear panel side edge;
the crotch region is located between the front panel and the rear panel, the crotch region being delimited in the longitudinal direction by the front panel crotch edge and the rear panel crotch edge;
the first front panel side edge is joined to the first rear panel side edge in the first side seam and the second front panel side edge is joined to the second rear panel side edge in the second side seam;
a front leg edge section of each of the first and the second leg edge is arranged in the elastically stretchable front body panel, a rear leg edge section of each of the first and the second leg edge is arranged in the elastically stretchable rear body panel, and a crotch leg edge section of each of the first and the second leg edge is arranged in the crotch region from the front panel crotch edge to the rear panel crotch edge, a total length of each leg edge being the combined length of the front leg edge section, the crotch leg edge section and the rear leg edge section;
the crotch leg edge section of the first and second leg edges extends partly or fully in the corresponding crotch elastic member;
the crotch leg edge section is shaped such that the intermediate portion of the crotch region has a smaller width than a width of the crotch region at the front panel crotch edge and a smaller width than a width of the crotch region at the rear panel crotch edge;
the width of the absorbent core in the intermediate portion of the absorbent core is smaller than a width of the absorbent core at the front panel crotch edge and smaller than a width of the absorbent core at the rear panel crotch edge.

4. A disposable absorbent article, according to claim 3, wherein the front end portions of each of the first and the second crotch elastic members overlap with and are attached to the front body panel and/or the rear end portions of the first and the second crotch elastic members overlap with and are attached to the rear body panel.

5. A disposable absorbent article according to claim 1, wherein the width of the front end portion of each crotch elastic member is equal to the width of the rear end portion of the crotch elastic member and a ratio between the width of the front and rear end portions and the smallest width of the intermediate portion is from 1:0.5 to 1:0.9.

6. A disposable absorbent article according to claim 1, wherein the crotch elastic members comprise or consist of elastic film or elastic nonwoven material.

7. A disposable absorbent article according to claim 1, wherein the absorbent core forms part of an absorbent assembly, the absorbent assembly comprising the absorbent core, a liquid permeable upper core cover layer and a lower core cover layer, the absorbent core being enclosed between the upper core cover layer and the lower core cover layer.

8. A disposable absorbent article according to claim 7, wherein the first side edges of the crotch elastic members are aligned with a first and a second side edge of the absorbent assembly and share a contour with the first and second side edges of the absorbent assembly in the crotch region.

9. A disposable absorbent article according to claim 8, wherein the upper core cover layer and the lower core cover layer are connected in a first side seal extending along the first side edge of the absorbent core and in a second side seal extending along the second side edge of the absorbent core, the absorbent core; comprising one or more channels extending in the longitudinal direction at a distance from each of the first side seal and the second side seal, each of the one or more channels having a channel seal extending therein, the channel seal joining the upper and lower layers of the core cover-s within the channel.

10. A disposable absorbent article according to claim 9, wherein a length of the one or more channels in the longitudinal direction is less than a length in the longitudinal direction of each of the first crotch elastic member and the second crotch elastic member.

11. A disposable absorbent article according to claim 9, wherein the first side seal, the second side seal and the channel seal or channel seals are adhesive seals or weld seals or combinations thereof.

12. A disposable absorbent article according to claim 11, wherein the first side seal, the second side seal and each channel seal are adhesive seals and a basis weight of adhesive in each channel seal is higher than a basis weight of adhesive in each of the first side seal and the second side seal.

13. A disposable absorbent article according to claim 7, wherein the upper core cover layer is a liquid permeable topsheet and the lower core cover layer is a liquid barrier layer.

14. A disposable absorbent article according to claim 1, wherein the absorbent core comprises super-absorbent material.

15. A disposable absorbent article according to claim 7, wherein the one or more channels are constituted by two channels extending spaced apart and symmetrically arranged on each side of the longitudinal centre line, between the longitudinal centre line and a corresponding one of the first side seal and the second side seal.

16. A disposable absorbent article according to claim 7, wherein the length of each of the one or more channels is in the range of from 50 mm to 400 mm.

17. A disposable absorbent article according to claim 1, wherein the absorbent core comprises two or more parts having different absorption capacity per unit area.

18. A disposable absorbent article according to claim 17, wherein a central part of the absorbent core being arranged between a first end part of the absorbent core and a second end part of the absorbent core, as seen in the longitudinal direction, has a greater absorption capacity per unit area than an absorption capacity per unit area of the first end part and a greater absorption capacity per unit area than an absorption capacity per unit area of the second end part.

19. A disposable absorbent article according to claim 18, wherein the central part of the absorbent core has a uniform basis weight and each of the first end part and the second end part of the absorbent core has a uniform basis weight, the central part of the absorbent core being delimited from each of the first end part and the second end part by a corresponding first transition zone >4) and a second transition zone.

20. A disposable absorbent article according to claim 19, wherein the first end part is a front end part and the first transition zone is a front transition zone having an extension in the longitudinal direction of from 5 millimetres to 80 millimetres and wherein the second end part is a rear end part and the second transition zone is a rear transition zone having an extension in longitudinal direction of from 5 millimetres to 80 millimetres.

21. A disposable absorbent article according to claim 19, wherein a ratio between the basis weight of the central part of the absorbent core and the basis weight of the first and the second end parts is in the range of from 4 to 1.5.

22. A disposable absorbent article according to claim 1, wherein the absorbent core has a length being determined as the distance between a point on the first end edge of the absorbent core where the first end edge of the absorbent core intersects with the longitudinal centre line and a point on the second end edge of the absorbent core where the second end edge intersects with the longitudinal centre line, the length of the absorbent core being in the range of from 300 millimetres to 700 millimetres.

23. A disposable absorbent article according to claim 1, wherein the crotch region comprises a non-stretchable nonwoven web.

24. A disposable article according to claim 3, wherein the elastically stretchable front body panel and/or the elastically stretchable rear body panel comprises or consists of an elastic laminate, the elastic laminate comprising at least a first layer of fibrous material and an elastic layer.

25. A disposable article according to claim 24, wherein the elastic layer comprises or consists of a layer of elastic film or a layer of elastic nonwoven.

26. A disposable absorbent article according to claim 1, wherein the absorbent core comprises from 50% by weight to 100% by weight of superabsorbent material.
